(19) United States
(12) Patent Application Publication
(10) Pub. No.: US 2013/0309779 A1
(43) Pub. Date: Nov. 21, 2013

(54) OPTICAL WAVEGUIDE MEASUREMENT SYSTEM AND METHOD FOR MEASURING GLYCATED HEMOGLOBIN

(71) Applicant: KABUSHIKI KAISHA TOSHIBA, Tokyo (JP)

(72) Inventors: Shingo KASAI, Kanagawa-ken (JP); Ichiro TONO, Kanagawa-ken (JP)

(73) Assignee: Kabushiki Kaisha Toshiba, Tokyo (JP)

(21) Appl. No.: 13/834,958

(22) Filed: Mar. 15, 2013

(30) Foreign Application Priority Data
May 16, 2012 (JP) 2012-112871

(51) Int. Cl. G01N 33/543

(52) U.S. Cl. CPC G01N 33/54373
USPC 436/501; 422/69

(57) ABSTRACT

According one embodiment, an optical waveguide measurement system includes a first optical waveguide immobilizing a first substance, the first substance being able to be specifically bound to glycated hemoglobin; a plurality of first magnetic microparticles immobilizing a second substance immobilized, the second substance being able to be specifically bound to the glycated hemoglobin at a first site different from a second site, and the first substance can be specifically bound to the glycated hemoglobin at the second site; a first magnetic field applying section provided above the first optical waveguide and being able to move at least one of the plurality of first magnetic microparticles by magnetic force; a first light source being able to inject light into the first optical waveguide; and a first light receiving element being able to receive light ejected from the first optical waveguide.

### CROSS-REFERENCE TO RELATED APPLICATIONS

This application is based upon and claims the benefit of priority from the prior Japanese Patent Application No. 2012-112871, filed on May 16, 2012; the entire contents of which are incorporated herein by reference.

### FIELD

Embodiments described herein relate generally to an optical waveguide measurement system and a method for measuring glycated hemoglobin.

### BACKGROUND

HbA1c (hemoglobin A1c) is produced when sugar in blood is irreversibly bound to hemoglobin after entering a red blood cell. HbA1c reflects the average blood sugar level in blood during the past one or two months. Thus, HbA1c is widely used as an index for diabetes diagnosis, such as a screening test for diabetes and monitoring the state of blood sugar control of a diabetes patient.

As a method for measuring HbA1c, a latex agglutination method and a protease-based method are known. The latex agglutination method is simple in operation. However, in the latex agglutination method, bulk turbidity is measured. Thus, the apparatus is large, and the sensitivity may be inferior to the other methods. On the other hand, in the protease-based method, only the glycated portion of HbA1c is cut out by protease and measured. Thus, the protease-based method entails complicated preprocessing. Recently, the high performance liquid chromatography (HPLC) method has been becoming standard. However, this method is expensive. Furthermore, this method also requires a large apparatus, and entails further complicated processing.

### BRIEF DESCRIPTION OF THE DRAWINGS

FIGS. 1A and 1B are schematic sectional views of an optical waveguide measurement system according to a first embodiment;

FIG. 2 is a conceptual view of HbA1c;

FIGS. 3A to 3C are schematic views of a magnetic microparticle;

FIGS. 4A to 4D are process views showing a method for measuring glycated hemoglobin in a sample solution;

FIG. 5 is a schematic sectional view of an optical waveguide measurement system according to a second embodiment;

FIGS. 6A to 6C are process views showing a method for measuring glycated hemoglobin in a sample solution according to the second embodiment;

FIG. 7 is a schematic sectional view of an optical waveguide measurement system according to a third embodiment;

FIGS. 8A to 8C are process views showing a method for measuring glycated hemoglobin in a sample solution according to the third embodiment; and

FIGS. 9A and 9B are schematic views of an optical waveguide measurement system according to a fourth embodiment.

### DETAILED DESCRIPTION

In general, according to one embodiment, an optical waveguide measurement system of an embodiment includes a first optical waveguide with a first substance immobilized on the first optical waveguide, the first substance being able to be specifically bound to glycated hemoglobin; a second substance being able to be specifically bound to the glycated hemoglobin at a first site different from a second site, the first substance can be specifically bound to the glycated hemoglobin at the second site; and a plurality of first magnetic microparticles with the second substance immobilized on the plurality of first magnetic microparticles. The optical waveguide measurement system further includes a first magnetic field applying section provided above the first optical waveguide and being able to move at least one of the plurality of first magnetic microparticles by magnetic force; a first light source being able to inject light into the first optical waveguide; and a first light receiving element being able to receive light ejected from the first optical waveguide.

Embodiments will now be described with reference to the drawings. In the following description, like members are labeled with like reference numerals, and the description of the members once described is omitted appropriately.

### First Embodiment

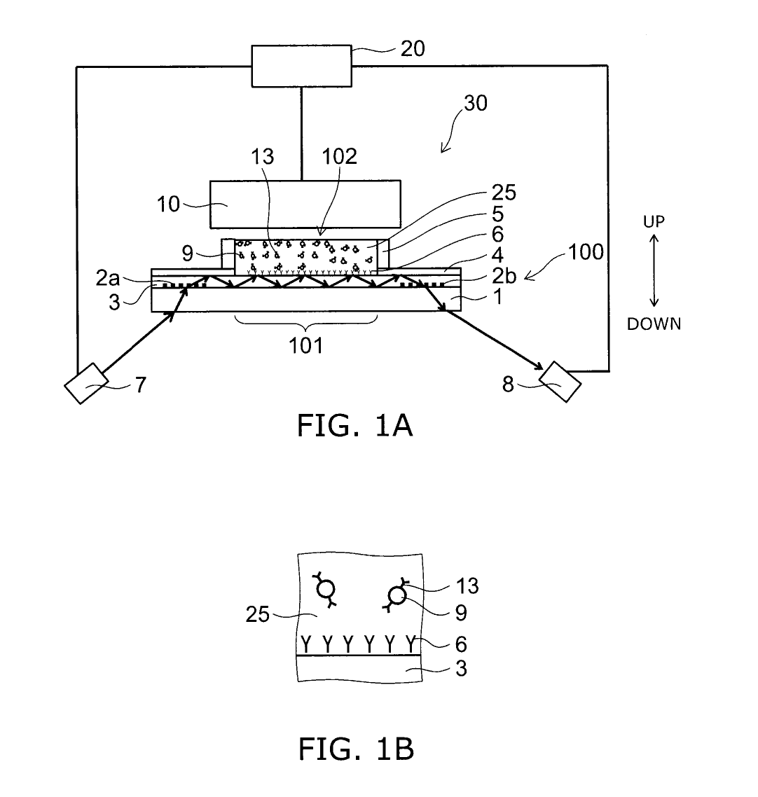

FIGS. 1A and 1B are schematic sectional views of an optical waveguide measurement system according to a first embodiment. FIG. 1A is a schematic sectional view showing the entirety of the optical waveguide measurement system. FIG. 1B is a schematic sectional view enlarging the neighborhood of a first substance immobilized on an optical waveguide of the optical waveguide measurement system.

The optical waveguide measurement system 30 according to the first embodiment includes an optical waveguide 3 (first optical waveguide), a first substance 6 immobilized in a prescribed region (the sensing area 101 described later) on the optical waveguide 3 and being able to be specifically bound to glycated hemoglobin, a second substance 13, a plurality of magnetic microparticles 9 (first magnetic microparticles), and a liquid (water, organic solvent) or other dispersion medium 25 (first dispersion medium). On each of the plurality of magnetic microparticles 9, the second substance 13 is immobilized. The second substance 13 can be specifically bound to glycated hemoglobin at a site different from the site where the first substance 6 can be specifically bound to glycated hemoglobin. The plurality of magnetic microparticles 9 are dispersed into the dispersion medium 25 and the dispersion medium 25 is in contact with the first substance 6.

The optical waveguide measurement system 30 further includes a magnetic field applying section 10 (first magnetic field applying section) provided above the optical waveguide 3 and being able to move at least one of the plurality of magnetic microparticles 9 in the dispersion medium 25 by magnetic force, a light source 7 (first light source) being able to inject light into the optical waveguide 3 from outside the aforementioned prescribed region, and a light receiving element 8 (first light receiving element) being able to receive light ejected from the optical waveguide 3 outside the aforementioned prescribed region. The light emitted from the light source 7 acts on the aforementioned prescribed region. The magnetic field applying section 10 can apply a magnetic field for moving at least one of the plurality of magnetic microparticles 9 in a direction away from the optical waveguide 3.

Besides, the optical waveguide measurement system 30 includes a substrate 1 for supporting the optical waveguide 3, gratings 2a, 2b (incoming side grating 2a and outgoing side grating 2b) provided in the optical waveguide 3, a protective film 4 for protecting the surface of the optical waveguide 3, and a frame 5 provided on the optical waveguide 3. The gratings 2a, 2b are formed from a material having a higher refractive index than the substrate 1. The optical waveguide 3 having a flat surface is formed on the major surface of the substrate 1 including the gratings 2a, 2b. The protective film 4 covers the optical waveguide 3 from above. The protective film 4 is e.g. a resin film having a low refractive index. The protective film 4 is provided with an opening exposing part of the surface of the optical waveguide 3 located between the gratings 2a, 2b. The opening can be shaped like e.g. a rectangle. The surface of the optical waveguide 3 exposed in this opening constitutes a sensing area 101. The space surrounded with the optical waveguide 3 and the frame 5 is filled with the dispersion medium 25. The portion filled with the dispersion medium 25 may be called a reaction space 102.

In the sensing area 101, the first substance 6 is immobilized on the optical waveguide 3. The sensing area 101 is exposed from the protective film 4. The frame 5 is formed on the protective film 4 so as to surround the sensing area 101. In the optical waveguide measurement system 30, the configuration including the optical waveguide 3, the first substance 6, and the plurality of magnetic microparticles 9 with the second substance 13 immobilized thereon is referred to as optical waveguide sensor chip 100. The optical waveguide sensor chip 100 is portable, being detached from the optical waveguide measurement system 30. Furthermore, the optical waveguide measurement system 30 includes a control section 20 for controlling each of the light source 7, the light receiving element 8, and the magnetic field applying section 10.

The optical waveguide 3 can be e.g. a planar optical waveguide. The material of the optical waveguide 3 is e.g. one of thermosetting resin, photosetting resin, and alkali-free glass. The thermosetting resin or photosetting resin is e.g. one of phenol resin, epoxy resin, and acrylic resin. Specifically, the material of the optical waveguide 3 is a material transmissive to prescribed light. Preferably, the material of the optical waveguide 3 is a material having a higher refractive index than the substrate 1.

The sensing area 101 is a detection surface. Here, the immobilization of the first substance 6 is based on e.g. hydrophobic interaction or covalent bonding with the surface of the optical waveguide 3 in the sensing area 101. For instance, the first substance 6 is immobilized on the sensing area 101 by hydrophobization based on a silane coupling agent. Alternatively, a functional group may be formed on the sensing area 101, and a suitable linker molecule may be applied to immobilize the first substance 6 by chemical bonding. Regarding an example of the first substance 6, in the case where glycated hemoglobin in the sample solution is an antigen, its antibody (primary antibody) can be used as the first substance 6.

The second substance 13 specifically reacts with glycated hemoglobin in the sample solution. The second substance 13 is immobilized on the surface of the magnetic microparticle 9 by e.g. physisorption or chemical bonding via a carboxyl group or amino group. The magnetic microparticles 9 with the second substance 13 immobilized thereon are dispersed and retained in the sensing area 101 with the first substance 6 immobilized thereon. To form dispersion and retention of the magnetic microparticles 9, for instance, a slurry containing the magnetic microparticles 9 and a water-soluble substance is applied to the sensing area 101 or a surface (not shown) opposed to the sensing area 101 and dried. Alternatively, the magnetic microparticles 9 may be dispersed in a liquid and retained in e.g. a space or container (not shown) different from the reaction space 102.

The light source 7 radiates light on the aforementioned optical waveguide sensor chip 100. The light source 7 is e.g. a red laser diode. The light injected from the light source 7 is diffracted by the incoming side grating 2a and propagated in the optical waveguide 3. Then, the light is diffracted by the outgoing side grating 2b and ejected. The light ejected from the outgoing side grating 2b is received by the light receiving element 8, and the optical intensity is measured. The light receiving element 8 is e.g. a photodiode. The intensity is compared between the injected light and the ejected light to measure the light absorptance. Thus, the amount of magnetic microparticles 9 is measured. Then, the antigen concentration in the sample solution is determined based on the measured amount of magnetic microparticles 9. The details on determining the antigen concentration in the sample solution based on the measured amount of magnetic microparticles 9 will be described later.

The magnetic field applying section 10 applies a magnetic field to the optical waveguide sensor chip 100. The magnetic field applying section 10 generates a magnetic field and applies the generated magnetic field to the optical waveguide sensor chip 100 to move the magnetic microparticles 9 in response to the magnetic field. The magnetic field applying section 10 is placed on the opposite side of the magnetic microparticles 9 from the side where the optical waveguide 3 is located. In the first embodiment, the magnetic field applying section 10 is placed above in FIG. 1. The magnetic field applying section 10 includes e.g. a magnet or electromagnet. Preferably, the magnetic field intensity is dynamically adjusted by current using an electromagnet. However, a ferrite magnet or the like may be used to adjust the magnetic field intensity by the strength of the magnet itself or the distance from the optical waveguide sensor chip 100.

For instance, a ferrite magnet is placed above the optical waveguide sensor chip 100. A spacer is interposed between the magnet and the optical waveguide sensor chip 100. Then, the magnetic field intensity can be adjusted by changing the thickness of the spacer. Alternatively, the relative position of the ferrite magnet and the optical waveguide sensor chip 100 can be changed using an actuator such as linear motor to adjust the magnetic field intensity.

In the case of using an electromagnet, a coil is placed on the opposite side of the magnetic microparticles 9 from the precipitation side (the side of the optical waveguide 3), and a current is applied to the coil. Then, the magnetic field intensity can be adjusted by changing the current value.

In the first embodiment, a magnetic field is applied to the magnetic microparticles 9 by the magnetic field applying section 10. Thus, magnetic microparticles 9 adsorbed on the sensing area 101 without antigen-antibody reaction can be stripped from the sensing area 101. Thus, the absorbance due only to the magnetic microparticles 9 bound to the sensing area 101 via glycated hemoglobin by antigen-antibody reaction can be measured. This can reduce the measurement error.

Here, the magnetic microparticles 9 are preferably superparamagnetic microparticles, which rapidly lose magnetization upon stopping the application of magnetic field. Thus, even if the magnetic microparticles 9 are agglutinated to each other by magnetization upon application of magnetic field, the magnetic microparticles 9 can be redispersed by stopping the application of magnetic field. For instance, even if a magnetic field is applied when there is no glycated hemoglobin in the sample solution, agglutinates of magnetic microparticles 9 may be produced and become less likely to be stripped from the sensing area 101. Such agglutinates of magnetic microparticles 9 cause measurement error. In this case, if the magnetic microparticles 9 are configured to be superparamagnetic, agglutination of the magnetic microparticles 9 can be suppressed. Thus, the occurrence of measurement error can be suppressed.

Furthermore, in order to further improve the redispersibility upon stopping the application of magnetic field, positive or negative charge may be provided on the surface of the magnetic microparticle 9. Alternatively, a dispersant such as surfactant may be added to the dispersion medium of the magnetic microparticles 9.

Furthermore, in the first embodiment, spontaneously precipitated magnetic microparticles 9 can be pulled back upward by the magnetic field applying section 10. By repeating the spontaneous precipitation of the magnetic microparticles 9 and the upward pullback by the magnetic field applying section 10, the sample solution and the magnetic microparticles 9 can be stirred. This promotes binding by antigen-antibody reaction between the magnetic microparticle 9 and the sensing area 101 via the antigen (glycated hemoglobin) contained in the sample solution. Thus, high detection sensitivity can be achieved in a shorter time. This can enhance the detection sensitivity in the case where the concentration of glycated hemoglobin is low.

If positive or negative charge is provided on the surface of the magnetic microparticle 9, or a dispersant such as surfactant is added, the magnetic microparticles 9 are redispersed more easily upon stopping the application of magnetic field. This further promotes stirring. Thus, the detection sensitivity is further improved.

It is known that hemoglobin (Hb) in glycated hemoglobin is located in a red blood cell and bound to oxygen in the lung, for instance. Among hemoglobins, HbA (hemoglobin A) is known as adult hemoglobin. HbA has a tetrameric structure composed of two α subunits and two β subunits. The β subunit is specific to HbA. The α subunit is composed of 141 amino acids, and the β subunit is composed of 146 amino acids. HbA (α2β2) has a molecular weight of approximately 64500.

Glycated hemoglobin is a hemoglobin with sugar such as Glc (glucose), Fru (fructose), Suc (sucrose), and Mal (maltose) bound thereto.

HbA1 (hemoglobin A1) is HbA in which e.g. Glc (glucose) or phosphorylated sugar is bound to its β chain. HbA1c (hemoglobin A1c) is HbA1 in which Glc (glucose) is bound to its β chain N-terminal (Val (valine)).

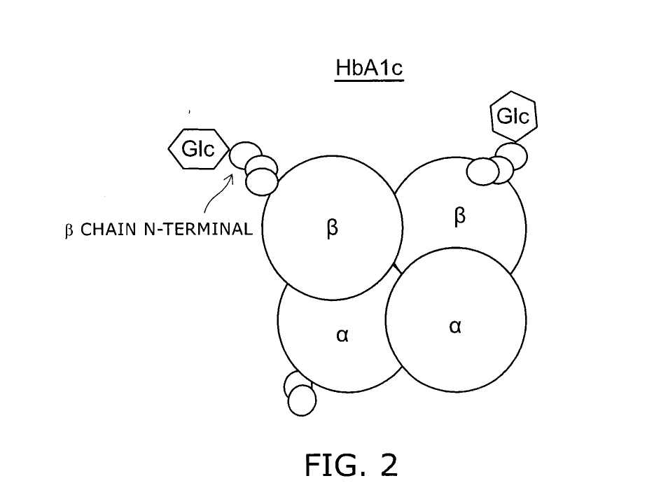

FIG. 2 is a conceptual view of HbA1c. As shown in FIG. 2, HbA1c includes two α subunits and two β subunits. In HbA1c, Glc (glucose) is bound to the β chain N-terminal of HbA1.

HbA1c is glycated in accordance with blood sugar, and accumulated until the lifetime of the red blood cell. Thus, the level of HbA1c is in proportion to the blood sugar level from the time of production of the red blood cell to the present, and reflects the average blood sugar level during the past one or two months.

As the first substance 6, a monoclonal antibody against an antigen which is the glycated peptide at the β chain N-terminal of HbA1c is selected. As the second substance 13, a monoclonal antibody against an antigen which is HbA1c other than the glycated peptide at the β chain N-terminal of HbA1c, or a monoclonal antibody against an antigen which is the β subunit of HbA1c other than the glycated peptide at the β chain N-terminal of HbA1c, is selected. The role of the first substance 6 and the second substance 13 will be described later.

The glycated peptide is a fructosyl peptide. More specifically, the glycated peptide is Fru (fructose)-Val (valine)-His (histidine)-Leu (leucine)-Thr (threonine)-Pro (proline)-Glu (glutamic acid).

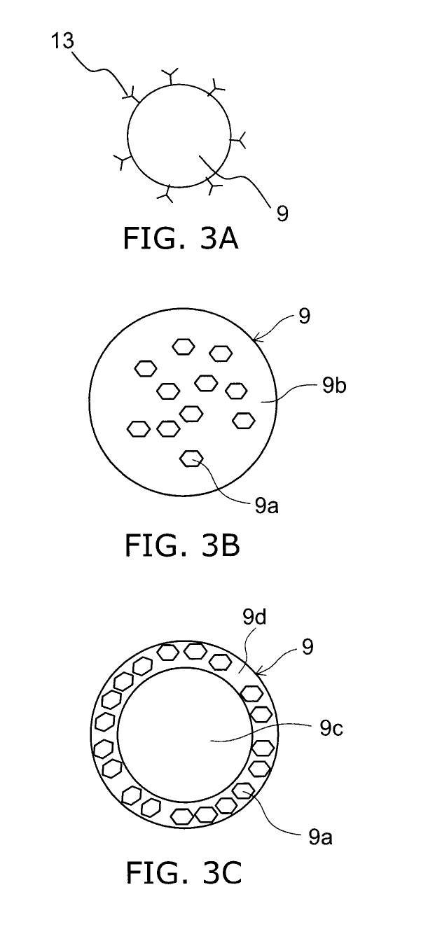

FIGS. 3A to 3C are schematic views of a magnetic microparticle. FIG. 3A is a schematic view for illustrating the appearance of the magnetic microparticle. FIGS. 3B and 3C are schematic sectional views for illustrating the cross section of the magnetic microparticle.

Before describing the specific structure of the magnetic microparticle 9, its overview is described.

The magnetic microparticles 9 are retained on the sensing area 101 in a dispersed state, or retained in e.g. a different space or container (not shown). Here, “the magnetic microparticles being retained on the sensing area in a dispersed state” means that the magnetic microparticles 9 are retained in a dispersed state directly or indirectly above the sensing area 101. An example configuration of “the magnetic microparticles being dispersed indirectly above the sensing area 101” is a configuration in which the magnetic microparticles 9 are dispersed via a blocking layer on the surface of the sensing area 101.

The blocking layer contains a water-soluble substance such as polyvinyl alcohol, bovine serum albumin (BSA), polyethylene glycol, phospholipid polymer, gelatin, casein, sugar (e.g., sucrose, trehalose), and synthetic polymer.

Another example configuration is a configuration in which the magnetic microparticles 9 are placed above the sensing area 101 with a space therebetween. For instance, a support plate (not shown) opposed to the sensing area 101 may be placed, and the magnetic microparticles 9 may be retained in a dispersed state on the surface of the support plate opposed to the sensing area 101.

In this case, preferably, the magnetic microparticles 9 are retained in a dry or semi-dry state. Preferably, the magnetic microparticles 9 are easily redispersed upon contact with the dispersion medium such as the sample solution. However, the configuration retained in a dry or semi-dry state does not necessarily need to be in a completely dispersed state. In the case of being retained in e.g. a different space or container, the dry or semi-dry state may be replaced by e.g. a state of being dispersed or precipitated in the dispersion medium.

The specific structure of the magnetic microparticle 9 is now described.

As shown in FIG. 3A, the second substance 13 is immobilized on the surface of the magnetic microparticle 9. The second substance 13 can be e.g. an antibody (secondary antibody) in the case where glycated hemoglobin in the sample solution is an antigen.

In this case, as shown in FIG. 3B, the magnetic microparticle 9 can be made of magnetic nanoparticles 9a covered with a polymer material 9b. Alternatively, as shown in FIG. 3C, the magnetic microparticle 9 can be configured to include a core 9c and a shell 9d covering the core 9c.

The core 9c can be formed from a polymer material. The shell 9d can be formed from a polymer material and configured to include magnetic nanoparticles 9a.

Alternatively, the magnetic microparticle 9 can be a microparticle itself made of a magnetic material. In this case, preferably, on the microparticle surface, the microparticle has a functional group for binding a recognition substance to be measured. The magnetic material used for the magnetic microparticle 9 can be e.g. any of various ferrites such as γ-Fe₂O₃. In this case, it is preferable to use a superparamagnetic material, which rapidly loses magnetism upon stopping the application of magnetic field.

In general, superparamagnetism is a phenomenon occurring in a nanoparticle of several ten nm (nanometers) or less. On the other hand, a microparticle causing light scattering needs to have a size of several hundred nm or more. Thus, a suitable magnetic microparticle 9 in the first embodiment is made of magnetic nanoparticles 9a covered with a polymer material 9b or the like as shown in FIG. 3B or 3C.

In general, the refractive index is mostly 1.5-1.6 for polymer materials, and approximately 3.0 for ferrites. When the magnetic microparticle 9 is located near the surface of the optical waveguide 3, the magnetic microparticle 9 having a higher refractive index is more likely to scatter light. Thus, it is considered that detection at higher sensitivity can be achieved when magnetic nanoparticles 9a having a higher refractive index are distributed near the surface of the magnetic microparticle 9.

As shown in FIG. 3B, in the magnetic microparticle 9 in which magnetic nanoparticles 9a are simply covered with a polymer material 9b, the magnetic nanoparticles 9a are distributed entirely in the microparticle. Thus, from the viewpoint of detection sensitivity, as shown in FIG. 3C, a suitable structure of the magnetic microparticle 9 is of the core-shell type in which magnetic nanoparticles 9a are included in the shell 9d at high density.

The particle diameter of the magnetic microparticle 9 is preferably 0.05 μm (micrometers) or more and 200 μm or less. If the particle diameter is smaller than 0.05 μm, the light scattering efficiency is decreased. If the particle diameter is larger than 200 μm, the efficiency of reaction between the second substance 13 and glycated hemoglobin may be decreased. Furthermore, despite application of magnetic force, the magnetic microparticle 9 may fail to be sufficiently moved due to the self-weight of the magnetic microparticle 9 itself. In order to further enhance the light scattering efficiency and reactivity, the particle diameter of the magnetic microparticle 9 is preferably set to 0.2 μm or more and 20 μm or less. Use of the particle diameter in this range enhances the light scattering efficiency and reactivity. This improves the detection sensitivity of the optical waveguide measurement system 30 for using light to detect glycated hemoglobin.

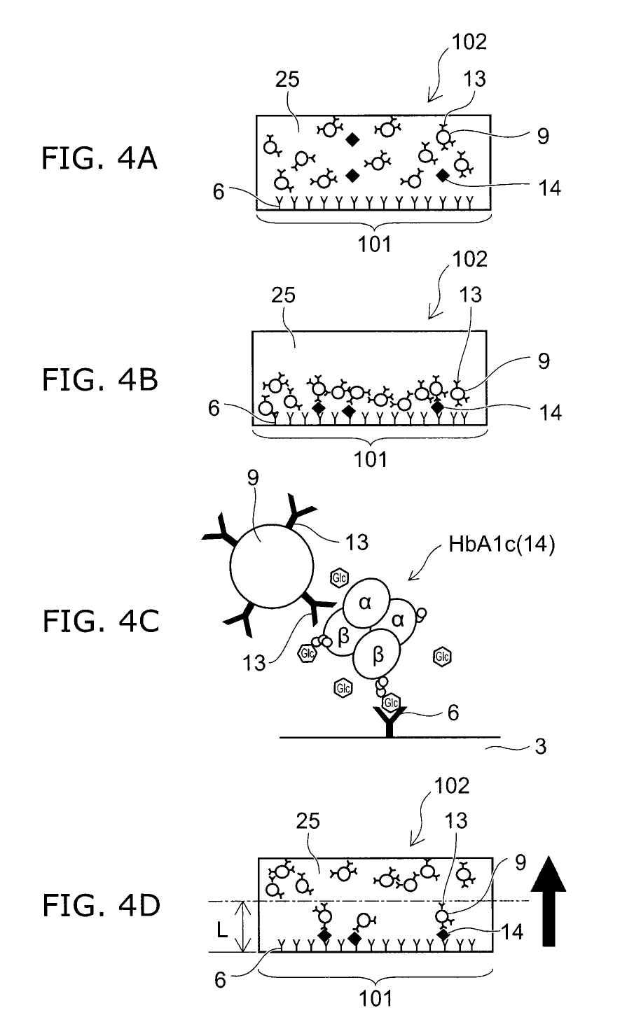

FIGS. 4A to 4D are process views showing a method for measuring glycated hemoglobin in a sample solution.

Here, the method for measuring the amount of glycated hemoglobin using the aforementioned optical waveguide measurement system 30 is described with reference to FIGS. 4A to 4D. As a glycated hemoglobin, HbA1c is selected. In FIGS. 4A to 4D, the state in the reaction space 102 is illustrated.

The procedure for measuring glycated hemoglobin in the sample solution starts with preparing a dispersion medium 25. The plurality of magnetic microparticles 9 are dispersed into this dispersion medium 25 The second substance 13 is immobilized on the plurality of magnetic microparticles 9. Furthermore, glycated hemoglobin is mixed in the dispersion medium 25. Next, the dispersion medium 25 is brought into contact with the first substance 6 on the optical waveguide 3. Next, the optical intensity of light ejected from the optical waveguide 3 is measured as first optical intensity. Next, a magnetic field is applied to the dispersion medium 25. Next, after the application of the magnetic field, the optical intensity of light ejected from the optical waveguide 3 is measured as second optical intensity. Then, the amount of glycated hemoglobin is determined based on the difference between the first optical intensity and the second optical intensity.

Specifically, first, as shown in FIG. 4A, on the optical waveguide 3 on which magnetic microparticles 9 are dispersed and retained, a sample solution is introduced to redisperse the magnetic microparticles 9. In the case where magnetic microparticles 9 are retained in e.g. a space other than on the optical waveguide 3 or a different container, a mixed dispersion liquid of the sample solution and magnetic microparticles 9 is introduced. Alternatively, a dispersion liquid of magnetic microparticles 9 and a sample solution may be separately introduced. The method of introduction can be e.g. dropping or pouring.

That is, the second substance 13 specifically bound to HbA1c (14) is immobilized on the magnetic microparticles 9. A dispersion medium 25 including HbA1c (14) and the magnetic microparticles 9 is brought into contact with the first substance 6 provided on the sensing area 101.

Next, as shown in FIG. 4B, the magnetic microparticles 9 are precipitated toward the sensing area 101 by self-weight. Then, the first substance 6 immobilized on the sensing area 101 is bound to the β chain N-terminal of HbA1c (14). The second substance 13 immobilized on the surface of the magnetic microparticle 9 is bound to e.g. the β subunit of HbA1c other than the glycated peptide at the β chain N-terminal of HbA1c. This state is shown in FIG. 4C. Thus, the magnetic microparticle 9 is bound to the sensing area 101 via HbA1c (14). At this stage, there are also magnetic microparticles 9 adsorbed on the sensing area 101 without the intermediary of HbA1c (14).

Next, as shown in FIG. 4D, a magnetic field is applied in a direction (e.g., upward) different from the precipitation direction as viewed from the magnetic microparticles 9. Thus, the magnetic microparticles 9 adsorbed on the sensing area 101 without the intermediary of HbA1c (14) are moved in the direction (e.g., upward) different from the precipitation direction and removed from the sensing area 101. That is, the magnetic microparticles 9 bound to the sensing area 101 via HbA1c (14) are left on the sensing area 101.

For instance, by adjusting the magnetic field intensity at an appropriate value, the magnetic microparticles 9 bound to the sensing area 101 via HbA1c (14) by antigen-antibody reaction are not stripped from the first substance 6. Thus, the magnetic microparticles 9 adsorbed on the sensing area 101 without the intermediary of HbA1c (14) can be removed from the sensing area 101.

In the first embodiment, it is considered that an appropriate magnetic field intensity can be determined as follows. The state of magnetic microparticles 9 can be detected by near-field light such as evanescent light. Such states can be classified into the following states A-C by the difference in the strength of interaction with the sensing area 101.

The states are listed in the order from the strongest interaction. State A is the state of magnetic microparticles 9 bound to the sensing area 101 by binding between HbA1c (14) and a molecule specifically bound thereto. State B is the state of magnetic microparticles 9 nonspecifically adsorbed on the sensing area 101 by intermolecular force or hydrophobic interaction. State C is the state of magnetic microparticles 9 floating near the sensing area 101. The magnetic microparticles 9 in state A are magnetic microparticles 9 which should contribute to detecting the concentration of HbA1c (14).

The magnetic microparticles 9 in state B or state C are magnetic microparticles 9 possibly causing measurement error (noise). Here, the magnetic microparticles 9 in state A are often referred to herein as magnetic microparticles 9 bound to the sensing area 101.

The magnetic microparticles 9 in state B are often referred to herein as magnetic microparticles 9 adsorbed on the sensing area 101.

The “surface neighborhood” of the optical waveguide 3 which can be detected by near-field light is now described. For instance, when light is propagated by total reflection, evanescent light seeps out on the surface of the propagating body. In the case of such evanescent light, the seeping distance d is determined by the following formula (1). It is found from formula (1) that the seeping distance d is approximately several times smaller than the wavelength of light used for measurement.

d=λ/{2π(n₁×sin² θ−n₂²)^{1/2}} (1)

Here, d is the seeping distance of evanescent light, λ is the wavelength of light used for measurement, n₁ is the refractive index of the optical waveguide 3, n₂ is the refractive index of the dispersion medium 25 dispersed with the magnetic microparticles 9, and θ is the total reflection angle.

Thus, the magnetic field applying section 10 applies a magnetic field having a magnetic field intensity such that the magnetic microparticles 9 are separated from the sensing area 101 by the distance L satisfying the following formula (2).

L>λ/{2π(n₁×sin² θ−n₂²)^{1/2}} (2)

Here, L is the distance by which the magnetic microparticles 9 are separated from the sensing area 101, λ is the wavelength of light used for measurement, n₁ is the refractive index of the optical waveguide 3, n₂ is the refractive index of the dispersion medium 25 dispersed with the magnetic microparticles 9, and θ is the total reflection angle.

For instance, L>130 nm when λ=635 nm, n₁=1.58, n₂=1.33 (in the case where the dispersion medium 25 is water), and θ=78°. Thus, by application of magnetic field, magnetic microparticles 9 in state B or state C are separated from the sensing area 101 by a slight distance of approximately several hundred nm. Simply by this separation, the measurement error can be sufficiently reduced. Thus, only a slight period of time is needed to separate magnetic microparticles 9 in state B or state C from the sensing area 101 by a distance enough to avoid the error of detection sensitivity.

Within an allowable range of time, although a longer time is needed, magnetic microparticles 9 in state B or state C can be moved with a weaker magnetic field intensity by a distance enough to avoid causing measurement error. This can reduce the possibility of excessively stripping the magnetic microparticles 9 needed for the measurement of state A. That is, the magnetic microparticles 9 in state A which should contribute to measurement are not stripped from the sensing area 101, and the magnetic microparticles 9 in state B or state C possibly constituting the noise of measurement can be stripped from the sensing area 101 beyond the distance affecting the measurement. This can improve the S/N ratio.

Thus, the appropriate magnetic field intensity is an appropriate magnetic field intensity in the sense that the magnetic microparticles 9 in state A which should contribute to measurement are not stripped from the sensing area 101, and the magnetic microparticles 9 in state B or state C possibly constituting the noise of measurement are stripped from the sensing area 101 beyond the distance affecting the measurement.

As described above, preferably, the magnetic field intensity is optimally adjusted by current using an electromagnet. However, a ferrite magnet or the like may be used to adjust the magnetic field intensity by changing the strength of the magnet itself or the relative position of the optical waveguide sensor chip 100 and the magnet. In the case of using an electromagnet, a coil is placed on the opposite side of the magnetic microparticles 9 from the precipitation side (the side of the optical waveguide 3), and a current is applied to the coil. Then, the magnetic field intensity can be adjusted by changing the current value.

In order to optimally adjust the magnetic field intensity, the optical waveguide measurement system 30 of the first embodiment may further include a control section 20 for controlling the magnetic field intensity of the magnetic field applied by the magnetic field applying section 10. By this control section 20, the aforementioned control can be performed to adjust the magnetic field intensity at an appropriate level. For instance, the magnetic field intensity can be adjusted so that the magnetic microparticles 9 in state A which should contribute to measurement are not stripped from the sensing area 101, and the magnetic microparticles 9 in state B or state C possibly constituting the noise of measurement can be stripped from the sensing area 101 beyond the distance affecting the measurement.

In the case of adjusting the magnetic field intensity as needed, by adjustment using the control section 20, the magnetic field intensity can be dynamically controlled. For instance, the control section 20 can be configured to control at least one of timing and duration of applying a magnetic field by the magnetic field applying section 10.

Then, by measuring the difference of detection signal intensity ratio in the light receiving element 8, the amount of HbA1c (14) (i.e., antigen concentration) in the sample solution can be measured. Specifically, in FIGS. 1A and 1B, laser light from the light source 7 is injected from the incoming side grating 2a into the optical waveguide 3. The light is propagated in the optical waveguide 3 to generate near-field light such as evanescent light near the surface (exposed surface in the sensing area 101). In this state, a mixed dispersion liquid of the sample solution and magnetic microparticles 9 is introduced on the sensing area 101. Immediately thereupon (FIG. 4A), the magnetic microparticles 9 are precipitated and reach the neighborhood of the sensing area 101, e.g., the evanescent light region (FIG. 4B). The magnetic microparticles 9 are involved in absorption and scattering of evanescent light. This attenuates the intensity of reflected light.

Then, laser light ejected from the outgoing side grating 2b is received by the light receiving element 8. As a result, the intensity of the ejected laser light decreases with the passage of time due to the influence of the bound magnetic microparticles 9. Subsequently, an upward magnetic field is applied by the magnetic field applying section 10. Then, magnetic microparticles 9 in state B or state C are moved to the outside of the evanescent light region (FIG. 4D). Thus, the received light intensity is recovered to a prescribed value. The received light intensity at this time is compared with the received light intensity in the state of FIG. 4A, i.e., immediately after the introduction of the mixed dispersion liquid. Thus, the result can be obtained as a numerical value, e.g., decrease ratio.

Furthermore, after introducing the sample solution or the like into the optical waveguide sensor chip 100 and before applying a magnetic field, the optical intensity of light ejected from the optical waveguide sensor chip 100 (corresponding to an example of the first optical intensity) is measured. Furthermore, after applying the magnetic field, the optical intensity of light ejected from the optical waveguide sensor chip 100 (corresponding to an example of the second optical intensity) is measured. Then, the amount of HbA1c (14) can be determined based on the difference between these optical intensities.

The decrease ratio of the intensity of laser light received in the light receiving element 8 depends on the amount of magnetic microparticles 9 bound to the sensing area 101 primarily by antigen-antibody reaction and the like. That is, the decrease ratio is in proportion to the antigen concentration in the sample solution involved in antigen-antibody reaction. Thus, the variation curve of the intensity of laser light with the passage of time is determined in a sample solution with known antigen concentration. The decrease ratio of the intensity of laser light in a prescribed time after application of an upward magnetic field for this variation curve is determined. Thus, a calibration curve representing the relationship between the antigen concentration and the decrease ratio of the intensity of laser light is previously prepared. Next, from the time for measuring a sample solution with unknown antigen concentration by the aforementioned method and the variation curve of the intensity of laser light, the decrease ratio of the intensity of laser light for a prescribed time is determined. By comparing this decrease ratio of the intensity of laser light with the aforementioned calibration curve, the antigen concentration in the sample solution can be measured.

Next, a more specific example in which the measurement of the first embodiment is performed by experiment is described. The following specific numerical values and materials are illustrative only, and the embodiment is not limited to these numerical values and materials.

In the experiment, on a translucent substrate 1 made of e.g. glass, a titanium oxide film having a refractive index of 2.2-2.4 was formed to a thickness of 50 nm by sputtering technique. By lithography and dry etching technique, gratings 2a, 2b were formed. On the substrate 1 with the gratings 2a, 2b formed thereon, an ultraviolet curable acrylic resin film having a film thickness of approximately 10 μm was formed by spin coating technique and ultraviolet irradiation to form an optical waveguide 3. The refractive index after curing is 1.58.

A protective film 4 was formed on the optical waveguide 3. The protective film 4 is a low refractive index resin film. The protective film 4 was formed by screen printing technique so as to surround an antibody immobilized region. The antibody immobilized region is a sensing area 101 including a region corresponding to the region above the gratings 2a, 2b. The refractive index of the protective film 4 after drying is 1.34. In order to form a liquid pool for retaining the sample solution and the like, a frame 5 made of resin was fixed by double-faced tape. On the surface of the region between the gratings where the protective film was not formed, the first substance 6 for HbA1c (14) was immobilized by covalent bonding technique.

In the first embodiment, the magnetic microparticle 9 was of the core-shell type in which the shell 9d includes magnetic nanoparticles 9a at high density. The average particle diameter of the magnetic microparticle 9 was set to 1.1 μm. A dispersion liquid including such magnetic microparticles 9 was separately prepared.

Next, from the incoming side grating 2a, light having a center wavelength of 635 nm from a light emitting diode 7 was injected. The optical intensity of light ejected from the outgoing side grating 2b was measured by a photodiode 8. Simultaneously, the sample solution and the dispersion liquid of the magnetic microparticles 9 were mixed and then introduced on the sensing area 101 (inside the frame 5). Then, measurement was performed in accordance with the aforementioned measurement procedure.

In the first embodiment, a ferrite magnet was placed above the optical waveguide sensor chip 100. A spacer was provided between the ferrite magnet and the optical waveguide sensor chip 100. By changing the thickness of the spacer, the magnetic field intensity was changed.

The photodiode detection signal intensity at this stage was compared with the intensity (initial intensity) immediately after introducing the mixed solution to measure the difference therebetween. The amount of attenuation of signal intensity indicated in this difference corresponds to the number of magnetic microparticles 9 bound to the surface of the optical waveguide 3 via HbA1c (14). Thus, the concentration of HbA1c (14) to be measured can be calculated.

Thus, the first substance 6 specifically reacting with HbA1c is immobilized on the optical waveguide 3. The second substance 13 is immobilized on the magnetic microparticle 9. By using the optical waveguide 3 and the magnetic microparticles 9, nonspecifically adsorbed noise particles of the magnetic microparticles 9 are removed by magnetic field application. By this method, HbA1c at ultralow concentration can be measured with high sensitivity without the need of binding-free separation (B/F separation).

Here, in the case of using an antibody against the substance to be measured, or in the case where the substance to be measured is an antibody, there are various immunoassay techniques as methods for measuring the concentration of the substance to be measured using an antigen or antibody against that antibody.

For instance, in enzyme immunoassay (EIA or ELISA), primary antibodies corresponding to the substance to be measured in the sample to be measured are immobilized on the surface of a well-shaped substrate. Then, a prescribed amount of sample solution is added into the well to perform primary reaction. Subsequently, a solution of secondary antibodies labeled with an enzyme catalyzing the chromogenic reaction of a dye is added to perform secondary reaction. Excess secondary antibodies are removed by washing. Then, a chromogenic reagent is added to measure the absorbance. However, the foregoing procedure is complicated.

As another immunoassay method with higher sensitivity, the CLEIA method is known. In the CLEIA method, secondary antibodies labeled with an enzyme catalyzing chemiluminescence reaction are used as secondary reaction to detect the amount of chemiluminescence. Furthermore, the immunochromatography method is known. In the immunochromatography method, the presence or absence of the substance to be measured can be determined simply by dropping a sample liquid.

However, in these immunoassay methods, secondary antibodies not bound to the substance to be detected constitute the background or noise component. Thus, the immunoassay methods in common require the process of sufficiently removing excess secondary antibodies by washing (binding-free separation (B/F separation) process). This results in a longer working time. Furthermore, automating these process steps leads to increasing the cost and size of the apparatus. On the other hand, the immunochromatography method is simple, but generally has low quantitative capability. In particular, the problem is that the determination in the low concentration region is varied depending on the measurer. Another problem is that the measurement result cannot be managed as digital values.

To solve these problems, the method for detecting microparticles bound to the surface by evanescent waves of the optical waveguide has been under study. This method does not need the B/F separation process. However, in the conventional method, actually, the process of binding microparticles to the optical waveguide surface by antigen-antibody reaction and the like is advanced by precipitation and Brownian diffusion of the microparticles, so to speak, in the natural course of events.

Thus, it is presumed that antigens contributing to binding of microparticles to the surface within a prescribed measurement time account for only a small fraction of all the antigens in the system. In addition, if the liquid property of the sample is varied, then the diffusion velocity, dispersion state and the like are varied accordingly. This causes additional measurement error. Furthermore, it is difficult to completely suppress the phenomenon of nonspecific physisorption of microparticles on the optical waveguide surface. This may constitute the noise component.

In another method for using evanescent waves, a first reactant is reacted with a second reactant. The first reactant has both a reacting portion specifically bound to the substance to be measured and a localization inducing portion. The second reactant has both a reacting portion specifically bound to the substance to be measured and a photoreacting portion. Then, the combined product of the first reactant, the substance to be measured, and the second reactant obtained by this reaction is localized in the near-field light region by localization means. Thus, the amount of the substance to be measured is determined by the difference of signals obtained before and after the reaction.

However, in the case where the first reactant is a magnetic microparticle, free first reactants not bound to antigens or second reactants are also simultaneously localized in the near-field light region. Furthermore, free second reactants exist in the near-field light region throughout the detection process. If the distribution of these reactants in the near-field light region is varied with e.g. the liquid property of the biological sample, this variation may constitute the noise component. In addition, no active countermeasures have been taken against the noise component due to nonspecific physisorption of the reactants and the combined products on the surface of detection means for generating near-field light.

As another method, an immunological sensor method using a Lamb wave mode acoustic device is known. In this sensor method, the labeling substance for detection is prepared as a latex or the like, and introduced into a reaction system. Then, the viscosity is varied accordingly. This causes phase difference change. Furthermore, the floating latex not adsorbed on the sensor portion causes noise.

To solve these problems, magnetic microparticles are used as the labeling substance. After reaction, a magnetic field is applied to distance the labeling substance floating near the sensor portion so that the environment around the acoustic device is made identical to that before inputting the labeling substance.

However, in the acoustic device in contact with liquid, the depth in the liquid coupled to the vibration of the sensor surface is considered to be several μm to several ten μm as determined from the following formula (3). The thickness d of the layer hydrodynamically coupled to the sensor surface is given by the following formula.

d=(2η/ωρ)^{1/2} (3)

(η: viscosity of liquid, ω: angular frequency, ρ: density of liquid)

Here, ω=2πF, where F is the frequency. Water has a viscosity of approximately 1 cp and a density of 1 g/cm³ (=103 kg/m³). Thus, if F is set to 5 MHz, d is approximately 8 μm.

Thus, when a magnetic field is applied after reaction so that the environment around the acoustic device is made identical to that before inputting the labeling substance, the labeling substance floating near the sensor portion needs to be distanced by approximately 10 μm or more. To reduce the measurement time, a stronger magnetic force needs to be applied. However, an excessively strong magnetic force also cuts the antigen-antibody bonding, and hence decreases the sensitivity.

In contrast, in the first embodiment, a magnetic field is applied to the magnetic microparticles 9 in a direction different from the precipitation direction. Thus, magnetic microparticles 9 adsorbed on the sensing area 101 without antigen-antibody reaction and the like and possibly constituting noise are stripped from the sensing area 101. Accordingly, the absorbance due to magnetic microparticles 9 bound to the sensing area 101 via glycated hemoglobin such as HbA1c by antigen-antibody reaction and the like can be measured. This can reduce the measurement error.

Furthermore, magnetic microparticles 9 possibly constituting noise can be removed by magnetic field application. This eliminates the need to remove such magnetic microparticles 9 by washing.

According to the first embodiment, the optical waveguide sensor chip 100 is used to perform measurement by near-field light such as evanescent light. This can reduce the distance by which magnetic microparticles 9 are stripped from the sensing area 101 beyond the range of affecting the measurement. Thus, the time required to strip the magnetic microparticles 9 from the sensing area 101 by an upward magnetic field can be made shorter. Alternatively, by a weaker magnetic field, the magnetic microparticles 9 can be stripped from the sensing area 101 beyond the range of affecting the measurement.

Furthermore, according to the first embodiment, the magnetic field intensity can be controlled. Thus, the magnetic microparticles 9 which should contribute to measurement are not stripped from the sensing area 101, and the magnetic microparticles 9 possibly constituting the noise of measurement can be stripped from the sensing area 101 beyond the distance affecting the measurement. This can improve the S/N ratio.

Furthermore, according to the first embodiment, the magnetic field intensity is dynamically controlled by the control section 20. Thus, high measurement accuracy can be maintained.

Furthermore, the magnetic microparticle 9 can be a superparamagnetic microparticle, which rapidly loses magnetization upon stopping the application of magnetic field. Then, the magnetic microparticles 9 are easily redispersed upon stopping the application of magnetic field. This suppresses the production of agglutinates of magnetic microparticles 9 even in the case where there is no glycated hemoglobin in the sample solution. Thus, the occurrence of measurement error can be suppressed.

Furthermore, the magnetic microparticle 9 can be of the core-shell type in which the shell 9d includes magnetic nanoparticles 9a. This can increase the scattering intensity of evanescent light. As a result, detection can be performed at high sensitivity.

Furthermore, positive or negative charge can be provided on the surface of the magnetic microparticle 9, or a dispersant such as surfactant can be added. Then, the magnetic microparticles 9 are redispersed more easily upon stopping the application of magnetic field. This can also reduce the measurement error.

Furthermore, according to the first embodiment, spontaneously precipitated magnetic microparticles 9 can be pulled back by applying a magnetic field in a direction different from the precipitation direction. By repeating the spontaneous precipitation of the magnetic microparticles 9 and the upward pullback by the magnetic field applying section 10, the sample solution and the magnetic microparticles 9 are stirred. This promotes antigen-antibody reaction between glycated hemoglobin (e.g., antigen) contained in the sample solution and the magnetic microparticles 9. Thus, high detection sensitivity can be achieved in a shorter time. This can enhance the detection sensitivity in the case where the concentration of glycated hemoglobin is low.

In this case, furthermore, positive or negative charge can be provided on the surface of the magnetic microparticle 9, or a dispersant such as surfactant can be added. Then, the magnetic microparticles 9 are redispersed more easily upon stopping the application of magnetic field. This can promote stirring and improve the detection sensitivity.

Furthermore, according to the first embodiment, the optical waveguide sensor chip 100 is used to measure the amount, concentration and the like of glycated hemoglobin by near-field light such as evanescent light. In this case, by using magnetic microparticles 9 having a particle diameter of 0.05 μm or more and 200 μm or less, or preferably 0.2 μm or more and 20 μm or less, the light scattering efficiency can be enhanced. Thus, the detection sensitivity of glycated hemoglobin can be improved.

### Second Embodiment

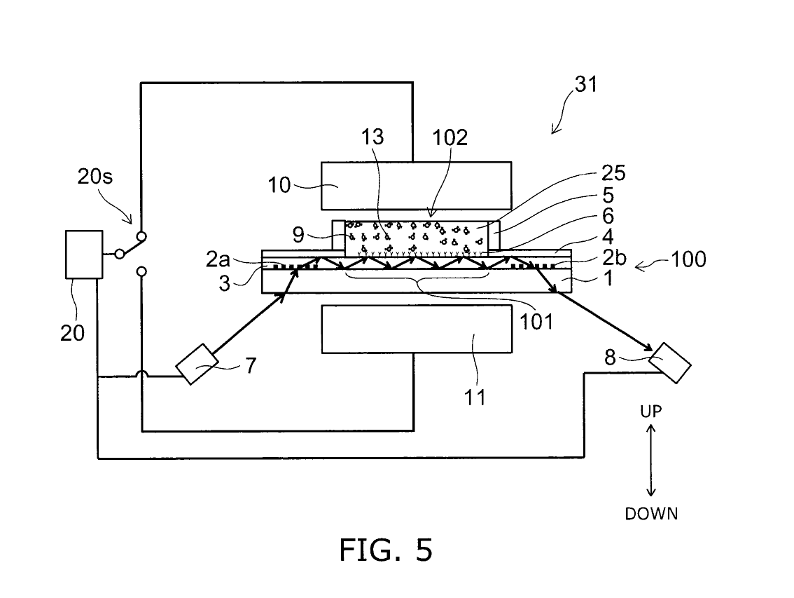

FIG. 5 is a schematic sectional view of an optical waveguide measurement system according to a second embodiment.

The optical waveguide measurement system 31 according to the second embodiment is different from the optical waveguide measurement system 30 of the first embodiment in further including a magnetic field applying section 11 (second magnetic field applying section). The magnetic field applying section 11 is provided below the optical waveguide 3. The rest of the configuration is similar to that of the first embodiment.

The magnetic field applying section 11 applies a magnetic field to the optical waveguide sensor chip 100 toward the optical waveguide 3 as viewed from the magnetic microparticles 9. The magnetic field applying section 11 can apply a magnetic field for moving at least one of the plurality of magnetic microparticles 9 in a direction toward the optical waveguide 3.

The magnetic field applying section 11 is provided on the side where the optical waveguide 3 is located as viewed from the magnetic microparticles 9. In the second embodiment, the magnetic field applying section 11 is provided below the sensor chip 100.

Like the magnetic field applying section 10, the magnetic field applying section 11 includes a magnet or electromagnet. Preferably, the magnetic field intensity is dynamically adjusted by current using an electromagnet. However, a ferrite magnet or the like may be used to adjust the magnetic field intensity by changing the strength of the magnet itself or the relative position of the optical waveguide sensor chip 100 and the magnet. For instance, a ferrite magnet is placed below the optical waveguide sensor chip 100. A spacer is interposed between the magnet and the optical waveguide sensor chip 100. Then, the magnetic field intensity can be adjusted by changing the thickness of the spacer. In the case of using an electromagnet, a coil is placed on the side of the optical waveguide 3 as viewed from the magnetic microparticles, and a current is applied to the coil. Then, the magnetic field intensity can be adjusted by changing the current value.

Here, the optical waveguide measurement system 31 of the second embodiment includes a control section 20. The control section 20 controls the intensity of the magnetic field applied to the sensing area 101 by at least one of the magnetic field applying section 10 and the magnetic field applying section 11. In this case, for instance, as shown in FIG. 5, a control section 20 common to the magnetic field applying section 10 and the magnetic field applying section 11, and a selector switch 20s can be provided. Alternatively, each of the magnetic field applying section 10 and the magnetic field applying section 11 can be provided with an independent control section. Alternatively, a control section for simultaneously controlling the magnetic field intensity of the magnetic field applying section 10 and the magnetic field applying section 11 can be provided. The control section 20 may be configured to dynamically optimize the magnetic field intensity by controlling the magnetic field intensity as needed.

The control section 20 may control the timing for applying a magnetic field in each of the magnetic field applying section 10 and the magnetic field applying section 11. Thus, the magnetic field applying section 10 and the magnetic field applying section 11 can alternately apply a magnetic field under a prescribed condition (e.g., a prescribed time instant, or a prescribed duration for keeping application of magnetic field, etc.).

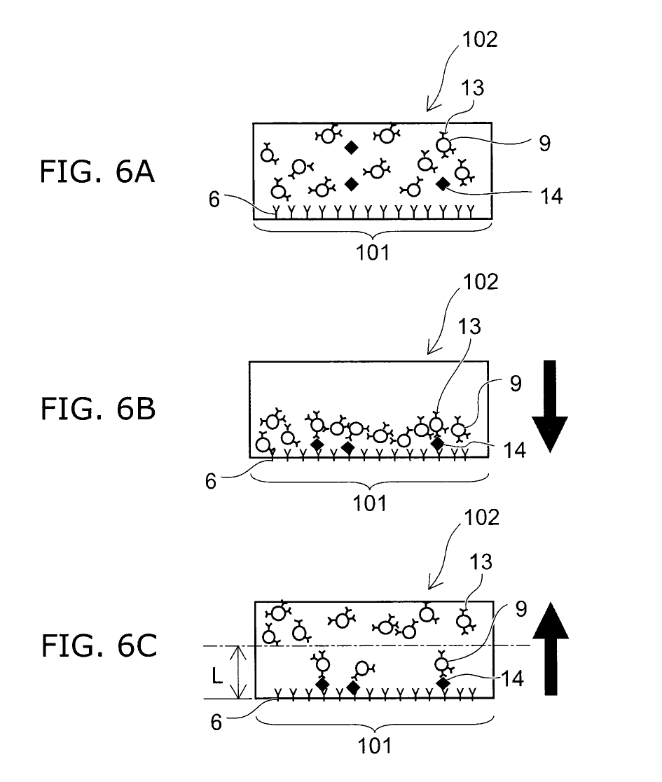

FIGS. 6A to 6C are process views showing a method for measuring glycated hemoglobin in a sample solution according to the second embodiment.

In FIGS. 6A to 6C, the state in the sensing area 101 is illustrated. The state of FIGS. 6A and 6C is similar to that of FIGS. 4A and 4D, and hence the description thereof is omitted.

By measuring the difference of detection signal intensity ratio in the light receiving element 8, the antigen concentration in the sample solution is measured. This is also similar to the first embodiment, and hence the description thereof is omitted.

The state of FIG. 6B is now described.

In FIG. 6B, in the precipitation direction (the direction toward the optical waveguide 3, e.g., downward in FIGS. 6A to 6C) as viewed from the magnetic microparticles 9, a downward magnetic field is applied by the magnetic field applying section 11. Thus, the magnetic microparticles 9 are attracted to the sensing area 101. Then, the first substance 6 (e.g., primary antibody) immobilized on the sensing area 101 is bound to the second substance 13 (e.g., secondary antibody) immobilized on the magnetic microparticle 9 via HbA1c (14) by antigen-antibody reaction. Thus, the magnetic microparticles 9 are bound to the sensing area 101.

In the second embodiment, the downward magnetic field application shown in FIG. 6B and the upward magnetic field application shown in FIG. 6C may be alternately repeated.

When the magnetic microparticles 9 are attracted to the optical waveguide 3 by the downward magnetic field application shown in FIG. 6B, in the sample solution, HbA1c (14) remains in the state of being not bound to any of the first substance 6 and the second substance 13. Alternatively, HbA1c (14) remains in the state of being bound to the second substance 13 immobilized on the surface of the magnetic microparticles 9, but not bound to the first substance 6 immobilized on the sensing area 101. Furthermore, on the sensing area 101, there are nonspecifically adsorbed magnetic microparticles 9.

Thus, in FIG. 6C, a magnetic field is applied with an intensity such that the magnetic microparticles 9 bound by antigen-antibody reaction and the like are not stripped. Accordingly, the magnetic microparticles 9 not bound by antigen-antibody reaction and the like are moved in a direction different from the direction toward the optical waveguide 3.

Subsequently, again, as shown in FIG. 6B, a magnetic field is applied in the direction toward the optical waveguide 3 to attract the magnetic microparticles 9 not bound by antigen-antibody reaction and the like. Then, HbA1c (14), and HbA1c (14) bound to the second substance 13 immobilized on the surface of the magnetic microparticles 9, are newly bound to the first substance 6 immobilized on the sensing area 101.

The foregoing is repeated. This decreases the number of magnetic microparticles 9 not bound to the sensing area 101 by antigen-antibody reaction and the like, and increases the number of magnetic microparticles 9 bound to the sensing area 101 by antigen-antibody reaction and the like. As a result, the S/N ratio is further improved.

The second embodiment also achieves an effect similar to that of the first embodiment. Furthermore, in the second embodiment, by repeating the downward magnetic field application and the upward magnetic field application at the time of initial precipitation, the rate of binding to the magnetic microparticles 9, the reaction efficiency, and the reproducibility are further improved.

For instance, according to the second embodiment, by applying a magnetic field to the magnetic microparticles 9 by the magnetic field applying section 11, the magnetic microparticles 9 can be attracted to the sensing area 101. Thus, the magnetic microparticles 9 are bound to the sensing area 101 more easily. This further improves the detection sensitivity of HbA1c (14).

Furthermore, after introducing the magnetic microparticles 9 and the sample solution into the reaction space 102, the magnetic microparticles 9 can be rapidly attracted toward the sensing area 101. This can reduce the time to wait for spontaneous precipitation of the magnetic microparticles 9. Thus, measurement can be performed within a short time. Furthermore, binding between the magnetic microparticle 9 and the sensing area 101 can be promoted before the progress of reaction and agglutination between the magnetic microparticles 9. This can further enhance the utilization ratio of HbA1c (14) for binding between the magnetic microparticle 9 and the sensing area 101. Thus, higher detection sensitivity is achieved.

Furthermore, the sample solution and the magnetic microparticles 9 can be stirred by moving the magnetic microparticles 9 by one or both of the magnetic field applying section 10 and the magnetic field applying section 11. This promotes antigen-antibody reaction and the like between HbA1c (14) contained in the sample solution and the magnetic microparticles 9. Thus, measurement with high detection sensitivity can be performed in a shorter time. Furthermore, further stirring can be performed by reciprocating the magnetic microparticles 9 by repeating the upward magnetic field application by the magnetic field applying section 10 and the downward magnetic field application by the magnetic field applying section 11.

This increases the opportunity for the magnetic microparticles 9 to be bound to the sensing area 101 via HbA1c (14). Thus, HbA1c (14) can be detected in a shorter time. Furthermore, the probability that the magnetic microparticles 9 are bound to the sensing area 101 can be increased, and the detection sensitivity and measurement accuracy of HbA1c (14) can be improved. For instance, this is effective in the case where the concentration of HbA1c (14) is low.

In the second embodiment, the magnetic microparticles 9 are stirred using a magnetic field. This eliminates the need of manual stirring operation and stirring mechanism having a pump and the like. Thus, a small measurement system easy to handle can be realized. For instance, if the magnetic field application by the control section 20 is automated, measurement can be performed by only one step of operation of a measurer introducing the sample solution into the sensor chip 100.

Furthermore, the magnetic microparticle 9 can be a superparamagnetic microparticle, which rapidly loses magnetization upon stopping the application of magnetic field. Then, even if the magnetic microparticles 9 are agglutinated to each other by magnetization upon application of magnetic field, the magnetic microparticles 9 can be redispersed by stopping the application of magnetic field. Even if the magnetic microparticles 9 are agglutinated to each other upon application of magnetic field, the application of magnetic field can be stopped before agglutinates of the magnetic microparticles 9 reach the neighborhood of the sensing area 101. Thus, the agglutinates of magnetic microparticles 9 can be redispersed. Accordingly, the magnetic microparticles 9 can reach the sensing area 101 in a dispersed state. This can prevent the increase of measurement noise due to the agglutination between the magnetic microparticles 9.

Furthermore, in order to further improve the redispersibility upon stopping the application of magnetic field, positive or negative charge may be provided on the surface of the magnetic microparticle 9. Alternatively, a dispersant such as surfactant may be added as a disperse medium to the surface of the magnetic microparticles 9.

Furthermore, according to the second embodiment, the magnetic field intensity of the magnetic field applying section and the magnetic field applying section 11 can be appropriately controlled by the control section 20 to improve the detection sensitivity and measurement accuracy of HbA1c (14).

### Third Embodiment

The first and second embodiments have been described with reference to the case where the optical waveguide is placed on the spontaneous precipitation side of the magnetic microparticles. In contrast, the third embodiment is described with reference to the configuration in which the optical waveguide is located on the opposite side from the spontaneous precipitation side of the magnetic microparticles.

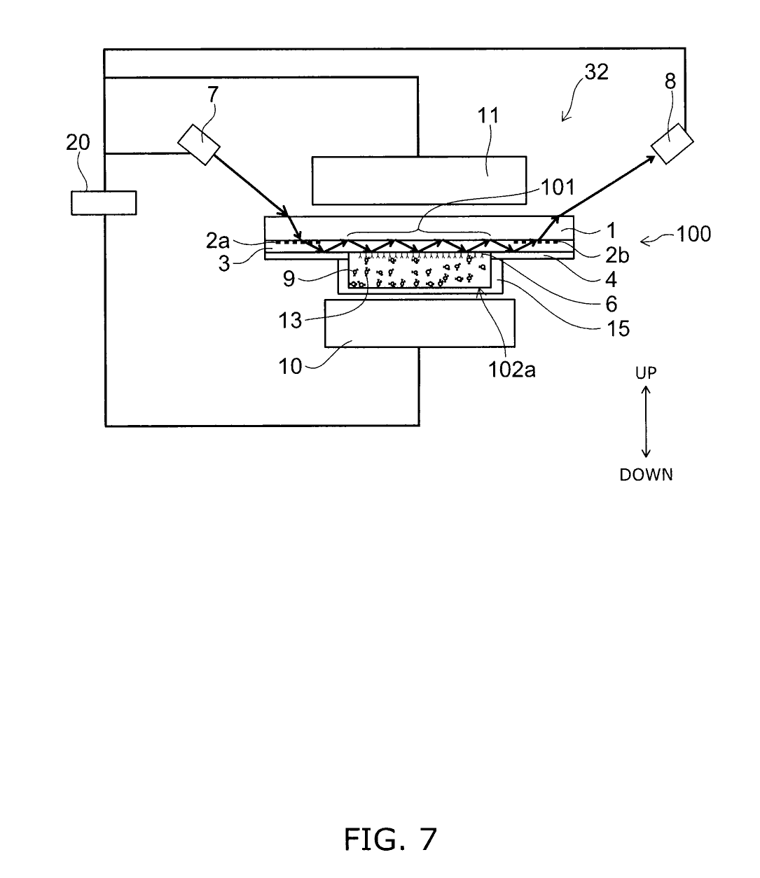

FIG. 7 is a schematic sectional view of an optical waveguide measurement system according to the third embodiment.

Instead of the frame 5 in the optical waveguide measurement system 31 of the second embodiment, the optical waveguide measurement system 32 according to the third embodiment uses a cap 15 shaped like an enclosure avoiding the drop of liquid, so that the entirety of the optical waveguide measurement system 31 of the second embodiment is vertically inverted. That is, in the third embodiment, the magnetic field applying section 10 is placed below the optical waveguide sensor chip 100, and the magnetic field applying section 11 is placed above the optical waveguide sensor chip 100. Thus, in the third embodiment, the magnetic field applying section 10 applies a downward magnetic field, and the magnetic field applying section 11 applies an upward magnetic field. The magnetic field applying section 10 is not necessarily needed. The rest of the configuration is similar to that of the second embodiment.

In order to retain a mixed dispersion liquid of the sample solution and magnetic microparticles 9, the optical waveguide measurement system 32 includes a cap 15 having e.g. a U-shaped cross section instead of the frame 5. The cap 15 and the sensing area 101 form a reaction space 102 constituting a semi-closed space except the opening for liquid introduction and an air vent hole (both not shown).

Here, the optical waveguide measurement system 32 of the third embodiment includes a control section 20. The control section 20 controls the intensity of the magnetic field applied to the sensing area 101 by at least one of the magnetic field applying section 10 and the magnetic field applying section 11. In this case, each of the magnetic field applying section 10 and the magnetic field applying section 11 may be provided with an independent control section. Alternatively, a control section common to the magnetic field applying section 10 and the magnetic field applying section 11, and a selector switch, not shown, can be provided. Alternatively, a control section for simultaneously controlling the magnetic field intensity of the magnetic field applying section 10 and the magnetic field applying section 11 can be provided. The magnetic field intensity may be dynamically optimized by controlling the magnetic field intensity as needed.

The control section 20 may control the timing for applying a magnetic field in each of the magnetic field applying section 10 and the magnetic field applying section 11. Thus, the magnetic field applying section 10 and the magnetic field applying section 11 can alternately apply a magnetic field to the dispersion medium 25 under a prescribed condition (e.g., a prescribed time instant, or a prescribed duration for keeping application of magnetic field, etc.).

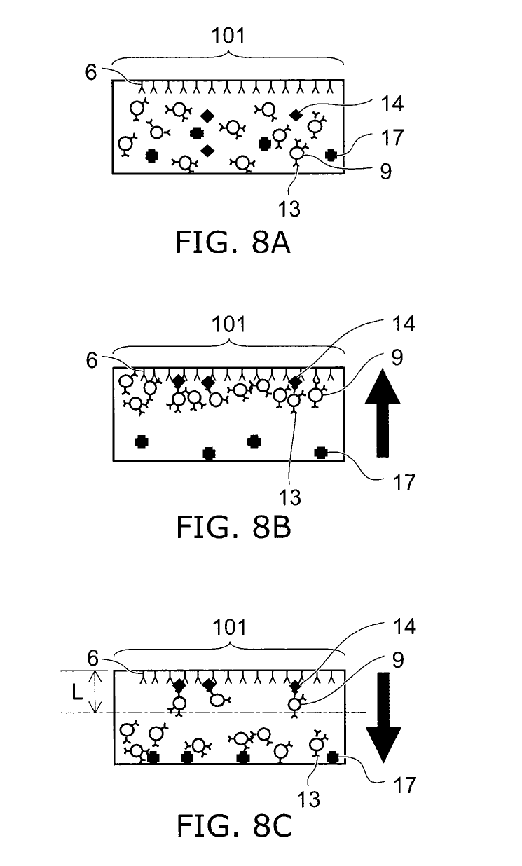

FIGS. 8A to 8C are process views showing a method for measuring glycated hemoglobin in a sample solution according to the third embodiment.

In FIGS. 8A to 8C, the state in the reaction space 102 is illustrated.

By measuring the difference of detection signal intensity ratio in the light receiving element 8, the amount and concentration (such as antigen concentration) of HbA1c (14) in the sample solution are measured. This is similar to the first embodiment, and hence the description thereof is omitted.

First, as shown in FIG. 8A, the reaction space 102 formed from the frame 5 and the sensing area 101 is filled with a mixed dispersion liquid of the sample solution and magnetic microparticles 9. The method for forming this state is similar to the method described in the first embodiment. Preferably, the introduction of the sample solution and the like is based on the method of pouring through the opening (not shown) for liquid introduction. Here, the sample solution may contain a foreign substance 17 precipitated by self-weight. The foreign substance 17 can be e.g. blood cell components in blood. If such a foreign substance 17 exists near the sensing area 101, the foreign substance 17 itself may act as a scatterer to cause measurement noise. Furthermore, the foreign substance 17 may prevent the reaction of binding the magnetic microparticles 9 to the sensing area 101 to decrease the measurement accuracy.

Next, as shown in FIG. 8B, in the direction toward the sensing area 101 as viewed from the magnetic microparticles 9, a magnetic field is applied by the magnetic field applying section 11. Thus, the magnetic microparticles 9 are attracted to the sensing area 101. Then, the first substance 6 (e.g., primary antibody) immobilized on the sensing area 101 is bound to the second substance 13 (e.g., secondary antibody) immobilized on the surface of the magnetic microparticle 9 via HbA1c (14) by antigen-antibody reaction. Thus, the magnetic microparticles 9 are bound to the sensing area 101. Simultaneously, the precipitating foreign substance 17 is moved downward in FIG. 8B (in the opposite direction from the sensing area 101) by self-weight.

Next, as shown in FIG. 8C, the downward magnetic field shown in FIG. 8C is applied by the magnetic field applying section 10. Then, magnetic microparticles 9 adsorbed on the sensing area 101 not by antigen-antibody reaction without the intermediary of HbA1c (14) are moved in the precipitation direction and removed from the sensing area 101. Here, also in a measurement system lacking the magnetic field applying section 10, simply by stopping the application of the upward magnetic field shown in FIG. 8B, the magnetic microparticles 9 adsorbed on the sensing area 101 not by antigen-antibody reaction and the like without the intermediary of HbA1c (14) could be moved downward by self-weight.

However, in this method, if the adsorption force of the magnetic microparticle 9 toward the sensing area 101 is greater than the downward force corresponding to the self-weight, it is difficult to remove the magnetic microparticles 9 adsorbed on the sensing area 101. Here, also in the process shown in FIG. 8C, the precipitating foreign substance 17 continues to move downward in FIG. 8B (in the opposite direction from the optical waveguide 3) by self-weight.

Also in the third embodiment, the upward magnetic field application shown in FIG. 8B and the downward magnetic field application or the precipitation of magnetic microparticles 9 by self-weight shown in FIG. 8C may be alternately repeated.

The third embodiment also achieves an effect similar to that of the first and second embodiments. Furthermore, according to the third embodiment, the sensing area 101 is located above the magnetic microparticles 9, and a magnetic field is applied to the magnetic microparticles 9 by the magnetic field applying section 11. Thus, simultaneously with attracting the magnetic microparticles 9 to the sensing area 101, the precipitating foreign substance 17 can be precipitated downward. Accordingly, the foreign substance 17 can be naturally moved to the outside of the evanescent light region near the sensing area 101. As a result, the measurement accuracy can be further enhanced without previously removing the foreign substance 17 by e.g. filtration.

### Fourth Embodiment

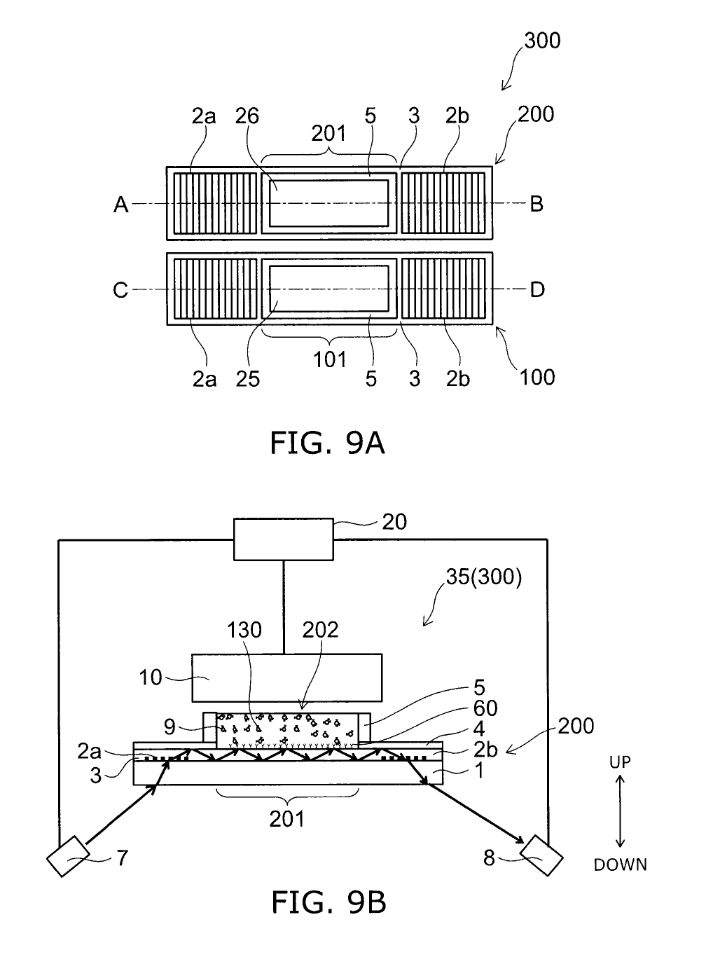

FIGS. 9A and 9B are schematic views of an optical waveguide measurement system according to a fourth embodiment. FIG. 9A is a schematic plan view, and FIG. 9B is a schematic sectional view at the position along line A-B of FIG. 9A.

The cross section at the position along line C-D in FIG. 9A corresponds to the cross section of the aforementioned optical waveguide measurement system 30. In FIG. 9A, the control section 20, the magnetic field applying section 10, the light source 7, and the light receiving element 8 described above are not shown. In FIG. 9A, the optical waveguide sensor chip is shown in plan view.

In the optical waveguide measurement system 300 according to the fourth embodiment, the aforementioned optical waveguide measurement system 30 and an optical waveguide measurement system 35 separate from the optical waveguide measurement system 30 are juxtaposed. In the optical waveguide measurement system 35, the concentration of total hemoglobin can be measured. That is, in the optical waveguide measurement system 300, hemoglobin antibodies are provided on one line, and HbA1c antibodies are provided on another line. Thus, the proportion of HbA1c in total hemoglobin can be calculated in one time of measurement. This requires representing the concentration of HbA1c as the concentration (%) for the amount of total hemoglobin. Thus, one of the two chips is used to measure the hemoglobin concentration, and the other is used to measure the HbA1c concentration.

The optical waveguide measurement system 35 according to the fourth embodiment includes an optical waveguide 3 (in the fourth embodiment, second optical waveguide), a third substance 60 immobilized on the optical waveguide 3 and being able to be specifically bound to hemoglobin, a fourth substance 130, the plurality of magnetic microparticles 9 (in the fourth embodiment, second magnetic microparticles), and a liquid (water, organic solvent) or other dispersion medium 25 (in the fourth embodiment, second dispersion medium). On each of the plurality of magnetic microparticles 9, the fourth substance 130 is immobilized. The fourth substance 130 can be specifically bound to hemoglobin at a site different from the site where the third substance 60 can be specifically bound to hemoglobin. The plurality of magnetic microparticles 9 are dispersed into the dispersion medium 25 and the dispersion medium 25 is in contact with the third substance 60.

The optical waveguide measurement system 30 includes a magnetic field applying section 10 (in the fourth embodiment, second magnetic field applying section) provided above the optical waveguide 3 and being able to move at least one of the plurality of magnetic microparticles 9 in the dispersion medium by magnetic force, a light source 7 (in the fourth embodiment, second light source) being able to inject light into the optical waveguide 3, and a light receiving element 8 (in the fourth embodiment, second light receiving element) being able to receive light ejected from the optical waveguide 3.

Besides, the optical waveguide measurement system 35 includes a substrate 1 for supporting the optical waveguide 3, gratings 2a, 2b (incoming side grating 2a and outgoing side grating 2b) provided in the optical waveguide 3, a protective film 4 for protecting the surface of the optical waveguide 3, and a frame 5 provided on the optical waveguide 3. The gratings 2a, 2b are formed from a material having a higher refractive index than the substrate 1. The optical waveguide 3 having a flat surface is formed on the major surface of the substrate 1 including the gratings 2a, 2b. The protective film 4 covers the optical waveguide 3 from above. The protective film 4 is e.g. a resin film having a low refractive index. The protective film 4 is provided with an opening exposing part of the surface of the optical waveguide 3 located between the gratings 2a, 2b. The opening can be shaped like e.g. a rectangle. The surface of the optical waveguide 3 exposed in this opening constitutes a sensing area 201. The space surrounded with the optical waveguide 3 and the frame 5 is filled with the dispersion medium 25. The portion filled with the dispersion medium 25 may be called a reaction space 202.

In the sensing area 201, the third substance 60 is immobilized on the optical waveguide 3. The sensing area 201 is exposed from the protective film 4. The frame 5 is formed on the protective film 4 so as to surround the sensing area 201. In the optical waveguide measurement system 35, the configuration including the optical waveguide 3, the third substance 60, and the plurality of magnetic microparticles 9 with the fourth substance 130 immobilized thereon is referred to as optical waveguide sensor chip 200. The optical waveguide sensor chip as a combination of the optical waveguide sensor chip 100 and the optical waveguide sensor chip 200 is portable. Furthermore, in the optical waveguide measurement system 35, each of the light source 7, the light receiving element 8, and the magnetic field applying section 10 is controlled by a control section 20.

The sensing area 201 is a detection surface. Here, the immobilization of the third substance 60 is based on e.g. hydrophobic interaction or covalent bonding with the surface of the optical waveguide 3 in the sensing area 201. For instance, the third substance 60 is immobilized on the sensing area 201 by hydrophobization based on a silane coupling agent. Alternatively, a functional group may be formed on the sensing area 201, and a suitable linker molecule may be applied to immobilize the third substance 60 by chemical bonding.

Regarding an example of the third substance 60, in the case where hemoglobin in the sample solution is an antigen, its antibody (primary antibody) can be used as the third substance 60.

The fourth substance 130 specifically reacts with hemoglobin in the sample solution. The fourth substance 130 is immobilized on the surface of the magnetic microparticle 9 by e.g. physisorption or chemical bonding via a carboxyl group or amino group. The magnetic microparticles 9 with the fourth substance 130 immobilized thereon are dispersed and retained in the sensing area 201 with the third substance 60 immobilized thereon. To form dispersion and retention of the magnetic microparticles 9, for instance, a slurry containing the magnetic microparticles 9 and a water-soluble substance is applied to the sensing area 201 or a surface (not shown) opposed to the sensing area 201 and dried. Alternatively, the magnetic microparticles 9 may be dispersed in a liquid and retained in e.g. a space or container (not shown) different from the reaction space 202.

Preferably, the third substance 60 and the fourth substance 130 are each a monoclonal antibody against an antigen which is an α subunit of hemoglobin, because total hemoglobin has α subunits in common. For instance, the third substance 60 is a monoclonal antibody against an antigen which is an α subunit of hemoglobin. For instance, the fourth substance 130 is a monoclonal antibody against an antigen which is an α subunit other than the α subunit of hemoglobin specifically reacting with the third substance 60. That is, the antigens of the third substance 60 and the fourth substance 130 are different α subunits of hemoglobin.

The optical waveguide measurement system 35 may include the aforementioned magnetic field applying section 11 besides the magnetic field applying section 10. Furthermore, the optical waveguide measurement system 35 may be vertically inverted as in FIG. 7. Furthermore, the optical waveguide measurement system 30 may be replaced by one of the optical waveguide measurement systems 31, 32.

The optical waveguide measurement system 300 as described above achieves an effect similar to that of the first to third embodiments. Furthermore, the proportion of HbA1c in total hemoglobin can be rapidly calculated by one time of measurement.

The embodiments have been described above with reference to examples. However, the embodiments are not limited to these examples. More specifically, these examples can be appropriately modified in design by those skilled in the art. Such modifications are also encompassed within the scope of the embodiments as long as they include the features of the embodiments. The components included in the above examples and the layout, material, condition, shape, size and the like thereof are not limited to those illustrated, but can be appropriately modified.

Furthermore, the components included in the above embodiments can be combined as long as technically feasible. Such combinations are also encompassed within the scope of the embodiments as long as they include the features of the embodiments. In addition, those skilled in the art could conceive various modifications and variations within the spirit of the embodiments. It is understood that such modifications and variations are also encompassed within the scope of the embodiments.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the invention.

What is claimed is:

1. An optical waveguide measurement system comprising:
a first optical waveguide immobilizing a first substance, the first substance being able to be specifically bound to glycated hemoglobin;
a plurality of first magnetic microparticles immobilizing a second substance immobilized, the second substance being able to be specifically bound to the glycated hemoglobin at a first site different from a second site, and the first substance can be specifically bound to the glycated hemoglobin at the second site,
a first magnetic field applying section provided above the first optical waveguide and being able to move at least one of the plurality of first magnetic microparticles by magnetic force;
a first light source being able to inject light into the first optical waveguide; and
a first light receiving element being able to receive light ejected from the first optical waveguide.

2. The system according to claim 1, wherein the glycated hemoglobin is HbA1c (hemoglobin A1c), and Glc (glucose) is bound to a β chain N-terminal of HbA1 (hemoglobin A1) in the HbA1c (hemoglobin A1c).

3. The system according to claim 2, wherein the first substance is a monoclonal antibody against an antigen, and the antigen is a glycated peptide at the β chain N-terminal of the HbA1c.

4. The system according to claim 3, wherein the second substance is a monoclonal antibody against an antigen, the antigen is HbA1c other than the glycated peptide at the β chain N-terminal of the HbA1c or a β subunit of the HbA1c other than the glycated peptide at the β chain N-terminal of the HbA1c.

5. The system according to claim 3, wherein the glycated peptide is a fructosyl peptide.

6. The system according to claim 1, wherein the first magnetic field applying section can apply a magnetic field for moving at least one of the plurality of magnetic microparticles in a direction away from the first optical waveguide.

7. The system according to claim 1, wherein the first magnetic field applying section can apply a magnetic field having a magnetic field intensity such that the plurality of magnetic microparticles are separated from the first substance by a distance L, the distance L satisfying a formula:
L>λ/{2π(n₁×sin² θn₂²)^{1/2}}
where L is the distance by which the plurality of magnetic microparticles are separated from the first substance, λ is wavelength of light used for measurement, n₁ is refractive index of the first optical waveguide, n₂ is refractive index of a dispersion medium for dispersing the plurality of magnetic microparticles, and θ is total reflection angle.

8. The system according to claim 1, further comprising:
a second magnetic field applying section below the first optical waveguide,
wherein the second magnetic field applying section can apply a magnetic field for moving at least one of the plurality of magnetic microparticles in a direction toward the first optical waveguide.

9. The system according to claim 8, wherein the first magnetic field applying section and the second magnetic field applying section can alternately apply a magnetic field to the dispersion medium.

10. The system according to claim 8, wherein at least one of the first magnetic field applying section and the second magnetic field applying section includes an electromagnet.

11. The system according to claim 8, further comprising:
a control section configured to control at least one of timing and duration for applying a magnetic field by at least one of the first magnetic field applying section and the second magnetic field applying section.

12. The system according to claim 11, wherein the control section can control magnetic field intensity of the magnetic field applied by at least one of the first magnetic field applying section and the second magnetic field applying section.

13. The system according to claim 1, wherein each of the plurality of magnetic microparticles includes a superparamagnetic material.

14. The system according to claim 1, wherein each of the plurality of magnetic microparticles includes a core and a shell covering the core, and the shell includes a magnetic nanoparticle.

15. The system according to claim 1, wherein each of the plurality of magnetic microparticles has positive or negative charge.

16. The system according to claim 1, wherein a surfactant is added to each of the plurality of magnetic microparticles.

17. An optical waveguide measurement system comprising:
an optical waveguide measurement system including:
a first optical waveguide with a first substance immobilized thereon, the first substance being able to be specifically bound to glycated hemoglobin;
a plurality of first magnetic microparticles with a second substance immobilized thereon, the second substance being able to be specifically bound to the glycated hemoglobin at a site different from a site where the first substance can be specifically bound to the glycated hemoglobin;
a first magnetic field applying section provided above the first optical waveguide and being able to move at least one of the plurality of first magnetic microparticles by magnetic force;
a first light source being able to inject light into the first optical waveguide; and
a first light receiving element being able to receive light ejected from the first optical waveguide; and
another optical waveguide measurement system including:
a second optical waveguide with a third substance immobilized thereon, the third substance being able to be specifically bound to hemoglobin;
a plurality of second magnetic microparticles with a fourth substance immobilized thereon, the fourth substance being able to be specifically bound to the hemoglobin at a site different from a site where the third substance can be specifically bound to the hemoglobin;
a second magnetic field applying section being able to move at least one of the plurality of magnetic microparticles by magnetic force;
a second light source being able to inject light into the second optical waveguide; and
a second light receiving element being able to receive light ejected from the second optical waveguide.

18. The system according to claim 17, wherein the third substance and the fourth substance are monoclonal antibodies against antigens, and each of the antigens is different α subunits of the hemoglobin.

19. The system according to claim 17, wherein the optical waveguide measurement system and the other optical waveguide measurement system are juxtaposed.

20. A method for measuring glycated hemoglobin,
a first substance being able to be specifically bound to the glycated hemoglobin, and the first substance being immobilized on a first optical waveguide,
a second substance being able to be specifically bound to the glycated hemoglobin at a first site different from a second site, the first substance can be specifically bound to the glycated hemoglobin at the second site, and the second substance being immobilized on a plurality of first magnetic microparticles, and
the plurality of first magnetic microparticles being dispersed into a first dispersion medium, the second substance being immobilized on the plurality of first magnetic microparticles, and the glycated hemoglobin being mixed into the first dispersion medium,
the method comprising:
bringing the first dispersion medium into contact with the first substance;
measuring optical intensity of light ejected from the first optical waveguide as first optical intensity;
applying a magnetic field to the first dispersion medium;
after the applying a magnetic field, measuring optical intensity of light ejected from the first optical waveguide as second optical intensity; and
determining amount of the glycated hemoglobin based on difference between the first optical intensity and the second optical intensity.
